(19) United States
(12) Patent Application Publication
(10) Pub. No.: US 2016/0136390 A1
(43) Pub. Date: May 19, 2016

(54) APPARATUSES AND METHODS FOR PERFORMING THERAPY ON TISSUE

(71) Applicant: Boston Scientific Scimed, Inc., Maple Grove, MN (US)

(72) Inventors: Aiden FLANAGAN, Kilcolgan (IE); Bryan Allen CLARK, Forest Lake, MN (US); Jason WEINER, Grafton, MA (US)

(73) Assignee: Boston Scientific Scimed, Inc.

(21) Appl. No.: 14/931,029

(22) Filed: Nov. 3, 2015

Related U.S. Application Data
(60) Provisional application No. 62/081,639, filed on Nov. 19, 2014.

(51) Int. Cl. A61M 25/01; A61B 18/14; A61B 18/24

(52) U.S. Cl. CPC A61M 25/01; A61B 18/24; A61B 18/1492; A61M 2205/3327; A61B 2018/00214

(57) ABSTRACT

An apparatus for positioning a device relative to tissue may include an emitter and a detector coupled to the device. The emitter may emit energy onto the tissue. The detector may detect energy reflected from the tissue. The detector may also generate a detector output signal indicative of a characteristic of the reflected energy. The apparatus may also include a processor that receives the detector output signal from the detector. The processor may determine whether there has been relative movement between the device and the tissue based on the detector output signal. The processor may also generate a processor output signal based on the relative movement. The apparatus may also include an actuator assembly engaging the device. The actuator assembly may receive the processor output signal from the processor. The actuator assembly may also move the device based on the processor output signal.

### CROSS-REFERENCE TO RELATED APPLICATION(S)

This patent application claims benefit of priority under 35 U.S.C. §119 to U.S. Provisional Patent Application No. 62/081,639, filed Nov. 19, 2014, the entirety of which is incorporated herein by reference.

### TECHNICAL FIELD

The present disclosure relates to apparatuses and methods for performing therapy on tissue. More particularly, the present disclosure relates to apparatuses and methods for positioning and/or stabilizing therapy devices relative to areas of tissue targeted for therapy.

### BACKGROUND

Chronic obstructive pulmonary disease (“COPD”) includes conditions such as, for example, chronic bronchitis and emphysema. COPD currently affects over 15 million people in the United States alone and is currently the third leading cause of death in the country. The primary cause of COPD is the inhalation of cigarette smoke, responsible for over 90% of COPD cases. The economic and social burden of the disease is substantial and is increasing.

Chronic bronchitis is characterized by chronic cough with sputum production. Due to airway inflammation, mucus hypersecretion, airway hyperresponsiveness, and eventual fibrosis of the airway walls, significant airflow and gas exchange limitations result.

Emphysema is characterized by the destruction of the lung parenchyma. This destruction of the lung parenchyma leads to a loss of elastic recoil and tethering which maintains airway patency. Because bronchioles are not supported by cartilage like the larger airways, they have little intrinsic support and therefore are susceptible to collapse when destruction of tethering occurs, particularly during exhalation.

Acute exacerbations of COPD (“AECOPD”) often require emergency care and inpatient hospital care. An AECOPD is defined by a sudden worsening of symptoms (e.g., increase in or onset of cough, wheeze, and sputum changes) that typically last for several days, but can persist for weeks. An AECOPD is typically triggered by a bacterial infection, viral infection, or pollutants, which manifest quickly into airway inflammation, mucus hypersecretion, and bronchoconstriction, causing significant airway restriction.

Despite relatively efficacious drugs (long-acting muscarinic antagonists, long-acting beta agonists, corticosteroids, and antibiotics) that treat COPD symptoms, a particular segment of patients known as “frequent exacerbators” often visit the emergency room and hospital with exacerbations and also have a more rapid decline in lung function, poorer quality of life, and a greater mortality risk.

Reversible obstructive pulmonary disease includes asthma and reversible aspects of COPD. Asthma is a disease in which bronchoconstriction, excessive mucus production, and inflammation and swelling of airways occur, causing widespread but variable airflow obstruction thereby making it difficult for the asthma sufferer to breathe. Asthma is further characterized by acute episodes of airway narrowing via contraction of hyper-responsive airway smooth muscle.

The reversible aspects of COPD include excessive mucus production and partial airway occlusion, airway narrowing secondary to smooth muscle contraction, and bronchial wall edema and inflation of the airways. Usually, there is a general increase in bulk (hypertrophy) of the large bronchi and chronic inflammatory changes in the small airways. Excessive amounts of mucus are found in the airways, and semisolid plugs of mucus may occlude some small bronchi. Also, the small airways are narrowed and show inflammatory changes.

In asthma, chronic inflammatory processes in the airway play a central role in increasing the resistance to airflow within the lungs. Many cells and cellular elements are involved in the inflammatory process including, but not limited to, mast cells, eosinophils, T lymphocytes, neutrophils, epithelial cells, and even airway smooth muscle itself. The reactions of these cells result in an associated increase in sensitivity and hyperresponsiveness of the airway smooth muscle cells lining the airways to particular stimuli.

The chronic nature of asthma can also lead to remodeling of the airway wall (i.e., structural changes such as airway wall thickening or chronic edema) that can further affect the function of the airway wall and influence airway hyper-responsiveness. Epithelial denudation exposes the underlying tissue to substances that would not normally otherwise contact the underlying tissue, further reinforcing the cycle of cellular damage and inflammatory response.

In susceptible individuals, asthma symptoms include recurrent episodes of shortness of breath (dyspnea), wheezing, chest tightness, and cough. Currently, asthma is managed by a combination of stimulus avoidance and pharmacology.

Bronchiectasis is a condition where lung airways become enlarged, flabby, and scarred. In the injured areas, mucus often builds up, causing obstruction and/or infections. A cycle of repeated infections may continue to damage the airways and cause greater mucus build-up. Bronchiectasis can lead to health problems such as respiratory failure, atelectasis, and heart failure.

Strategies for managing COPD and other conditions of the lung include smoking cessation, vaccination, rehabilitation, drug treatments (e.g., inhalers or oral medication), and delivering energy to airways. The energy delivered to the airway may heat portions of the wall of the airway. The application of heat to the airway wall may reduce the amount of excessive airway smooth muscle present in the airways and limit its ability to constrict and narrow the airway. Denervation and nerve stimulation therapies for the bronchial tree have been proposed to reduce bronchial hyperresponsiveness and the probability of AECOPD events by, for example, reducing the neural response of bronchial tree nerves, and may be carried out by delivering the energy to innervated tissue in the airway wall.

For some procedures, it may be beneficial to deliver energy to the airway wall, or any other body lumen wall, at a precise location, ensuring consistent energy delivery per tissue area per time. However, natural body motion due to breathing and heartbeats can move the position of an energy delivery device and/or the body lumen wall in any of a number of directions. This can cause a large variance in the energy delivered in a location and, therefore, cause a large variation in the therapeutic effect between patients and between locations in the body lumen.

Accordingly, a need exists for delivering energy and/or other types of therapy to areas of tissue, such as walls of body lumens, in a precise manner.

### SUMMARY

Aspects of the present disclosure relate to, among other things, apparatuses and methods for performing therapy on tissue. Each of the apparatuses and methods of the present disclosure may include one or more of the aspects described in connection with any of the other apparatuses and methods.

According to aspects of the present disclosure, an apparatus for positioning a device relative to tissue may include an emitter coupled to the device. The emitter may be configured to emit energy onto the tissue. The apparatus may also include a detector coupled to the device. The detector may be configured to detect energy reflected from the tissue. The detector may also be configured to generate a detector output signal indicative of a characteristic of the reflected energy. The apparatus may also include a processor configured to receive the detector output signal from the detector. The processor may also be configured to determine whether there has been relative movement between the device and the tissue based on the detector output signal. The processor may also be configured to generate a processor output signal based on the relative movement. The apparatus may also include an actuator assembly engaging the device. The actuator assembly may be configured to receive the processor output signal from the processor. The actuator assembly may also be configured to move the device based on the processor output signal.

In addition or alternatively, the device may include one or more other features. For example, the emitter may include a source of light configured to project light onto the tissue. The detector may include an imaging sensor configured to detect the energy reflected off of the tissue. The detector output signal may be indicative of a characteristic of the energy reflected off of the tissue. The detector output signal may be one of a plurality of detector output signals. The detector output signals may include a first detector output signal indicative of the characteristic of the energy reflected off of the tissue at a first point in time, and a second detector output signal indicative of the characteristic of the energy reflected off of the tissue at a second point in time. The processor may be configured to receive the first and second detector output signals. The processor may determine whether there has been relative movement between the device and the tissue by comparing the first and second detector output signals. The actuator assembly may be configured to move the device to compensate for the relative movement between the device and the tissue. The actuator assembly may be configured to move the device in synchronization with movement of the tissue.

According to aspects of the present disclosure, an apparatus may include a sheath having a lumen. The apparatus may also include a device. The device may include a member having a proximal portion and a distal portion. The member may be configured for insertion into the lumen of the sheath. The device may also include an end effector at the distal portion of the member. The device may also include an emitter configured to emit energy. The device may also include a detector configured to detect reflected energy and generate a detector output signal indicative of a characteristic of the reflected energy. The apparatus may also include a processor configured to receive the detector output signal from the detector. The processor may also determine whether the end effector has moved relative to adjacent tissue based on the detector output signal. The processor may also generate a processor output signal based on the movement. The apparatus may also include an actuator assembly engaging the member. The actuator assembly may be configured to receive the processor output signal from the processor. The actuator assembly may also move the device based on the processor output signal.

In addition or alternatively, the device may include one or more other features. For example, the end effector may include an array of electrically-conductive legs configured to delivery electrical energy to tissue. The end effector may include at least one of an inflatable balloon, a plurality of protrusions on at least one of the electrically-conductive legs, and a non-conductive leg. The end effector may include one of a biopsy instrument, a valve, and a stent. The actuator assembly may be configured to compensate for movement of the device. At least one of the emitter and the detector may be mounted on an external surface of the device. The emitter may be configured to emit energy onto a surface. The detector may be configured to detect the emitted energy after reflection off of the surface. The actuator assembly may be configured to synchronize movement of the device with movement of the surface.

According to aspects of the present disclosure, a method for positioning a device relative to tissue may include emitting energy toward the tissue with an emitter. The method may also include detecting reflected energy from the tissue with a detector. The method may also include generating a detector output signal indicative of a characteristic of the reflected energy. The method may also include receiving the detector output signal at a processor. The method may also include determining a position of the device relative to the tissue based on the detector output signal using the processor. The method may also include generating a processor output signal with the processor based on the position. The method may also include receiving the processor output signal at an actuator assembly. The method may also include moving the device relative to the tissue with the actuator assembly based on the processor output signal.

In addition or alternatively, the device may include one or more other features. For example, the step of determining the position of the device relative to the tissue may include identifying relative movement between the device and the tissue. The step of moving the device relative to the tissue with the actuator assembly may include moving the device in synchronization with the tissue. The step of moving the device relative to the tissue may include moving the device with the actuator assembly in first and second modes, the first mode including a first movement performed at a greater speed than a second movement performed in the second mode. The step of emitting energy toward the tissue may include emitting energy toward an identification element on a surface of the tissue. The step of detecting the reflected energy may include receiving the reflected energy after reflection off of the identification element. The step of moving the device relative to the tissue may include automatically moving the device relative to the tissue in a predetermined pattern using the actuator assembly.

It may be understood that both the foregoing general description and the following detailed description are exemplary and explanatory only and are not restrictive of the claimed features.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1A is a side view of a distal portion of an apparatus for performing therapy on tissue, in accordance with aspects of the present disclosure.

FIG. 1B, is a side view of a proximal portion of an apparatus for performing therapy on tissue, in accordance with aspects of the present disclosure.

FIG. 1C is a schematic illustration of a portion of an apparatus for performing therapy on tissue, in accordance with aspects of the present disclosure.

FIG. 2A is a side view of a distal portion of an apparatus for performing therapy on tissue, in accordance with aspects of the present disclosure.

FIG. 2B is a front view of a distal portion of an apparatus for performing therapy on tissue, in accordance with aspects of the present disclosure.

FIG. 2C is a side view of a distal portion of an apparatus for performing therapy on tissue, in accordance with aspects of the present disclosure.

FIG. 2D is a side perspective view of a distal portion of an apparatus for performing therapy on tissue, in accordance with aspects of the present disclosure.

FIG. 2E is a side perspective view of a distal portion of an apparatus for performing therapy on tissue, in accordance with aspects of the present disclosure.

FIG. 2F is a side perspective view of a distal portion of an apparatus for performing therapy on tissue, in accordance with aspects of the present disclosure.

FIG. 3 is a side perspective view of a device for performing therapy on tissue, in accordance with aspects of the present disclosure.

### DETAILED DESCRIPTION

Reference will now be made in detail to exemplary aspects of the present disclosure, examples of which are illustrated in the accompanying drawings. Wherever possible, the same reference numbers will be used throughout the drawings to refer to the same or like parts.

### Overview

The present disclosure is directed to apparatuses and methods for positioning devices relative to areas of tissue so therapies may be performed on the areas of tissue using the devices. The devices may include, for example, an energy-delivery device, a biopsy instrument, a valve delivery device, a stent delivery device, a diagnostic device, an imaging device, or any endoscopic or urologic device, or any other device for treatment of tissue defining any body lumen or cavity.

One type of therapy that may be performed, using the energy-delivery device, may entail using the device to apply energy to an area of tissue in a body lumen, such as in an airway of a lung. Accurate positioning of the device relative to the area of tissue may ensure consistent energy delivery to the area. Accurate positioning may be difficult when the device is guided manually to the area. Moreover, natural body motion due, for example, to breathing and/or heartbeats, can hinder accurate positioning of the device and/or move the device out of position. Such movement, if left uncorrected, may cause energy to be delivered to other areas that are not targeted for the therapy, and/or may cause a variance in the energy delivered per tissue area per time, decreasing the effectiveness of the therapy. Increasing the accuracy of positioning of the device and/or decreasing unwanted movement of the device relative to the tissue, may increase the effectiveness of the therapy.

Other types of therapy that may be performed may include performing a biopsy at an area of tissue, or deploying a valve or stent at an area of tissue. Accurate positioning of the biopsy instrument, valve, or stent relative to the area of tissue may be difficult when the biopsy instrument, valve, or stent is guided manually. Moreover, natural body motion due, for example, to breathing and/or heartbeats, can hinder accurate positioning of the biopsy instrument, valve, or stent, and/or move the biopsy instrument, valve, or stent out of position. Increasing the accuracy of positioning of the biopsy instrument, valve, or stent may enhance its performance.

### Exemplary Aspects

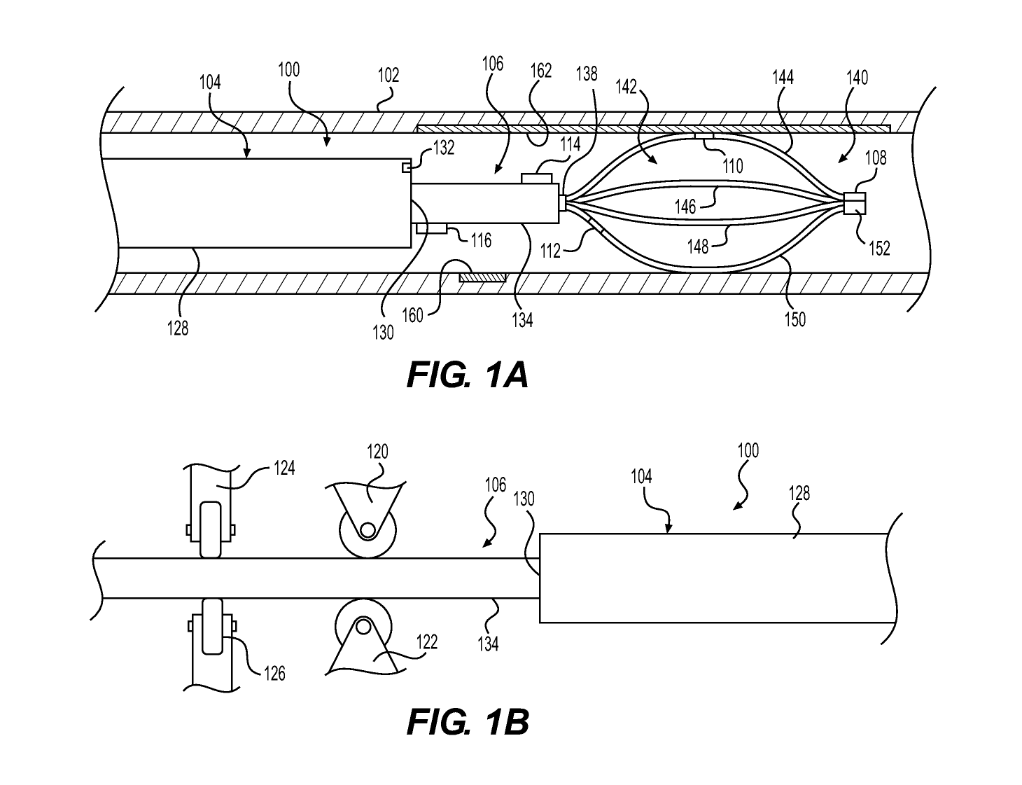

FIG. 1A shows a side view of a distal portion of an apparatus 100 for performing therapy on an area of tissue. The area of tissue may define a body lumen 102, such as in an airway of a lung, a blood vessel, or any other body lumen. Apparatus 100 may include an endoscope 104 and a therapy device 106. Apparatus 100 may also include one or more sensor assemblies 108, 110, 112, 114, 116 and one or more actuator assemblies 120, 122, 124, 126 (FIG. 1B).

Endoscope 104 may include an elongate member or sheath 128. A working channel 130 may extend through elongate member 128. A distal portion of elongate member 128 may be configured for insertion into a body and navigation through the body to an area of tissue. The area of tissue may define body lumen 102. It is also contemplated that endoscope 104 may include an imaging device 132, for example, at its distal tip by which a user may view an area of body lumen 102 distal to the distal tip.

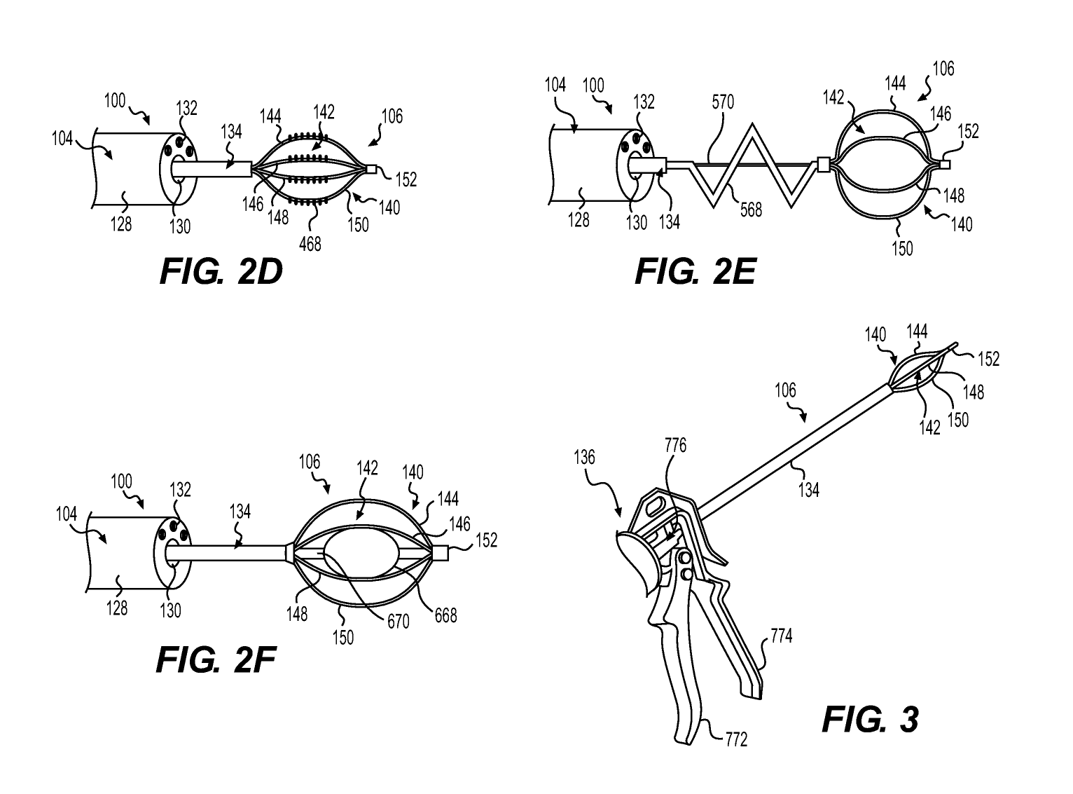

A distal portion of therapy device 106 may be configured for insertion through working channel 130. Therapy device 106 may include an elongate member or sheath 134. A shaft 138 may extend through elongate member 134. A handle 136 (FIG. 3) may be at a proximal end of elongate member 134. Handle 136 may be coupled to the proximal end of shaft 138. An end effector 140 may be at and/or coupled to a distal end of elongate member 134. Additionally or alternatively, end effector 140 may be coupled to a distal end of shaft 138, and slidable relative to elongate member 134. End effector 140 may be extended distally out of working channel 130 and/or elongate member 134.

End effector 140 may include an electrode array 142. Electrode array 142 may include one or more electrodes 144, 146, 148, 150. While four electrodes 144, 146, 148, 150 are described, it should be understood that fewer than four or more than four may be used. A proximal end of electrode 144 may be coupled to shaft 138. A distal end of electrode 144 may be coupled to an end cap 152. A proximal portion of electrode 144 and/or a distal portion of electrode 144 may be coated or otherwise covered with an insulating material (not shown), leaving only a portion of electrode 144, for example, the intermediate portion, exposed. Electrodes 146, 148, 150 may be similar to electrode 144. Electrodes 144, 146, 148, 150 may form legs of a basket.

As illustrated in FIG. 3, handle 136 may include actuation members 772, 774 and a locking mechanism 776. By exerting a force on or otherwise actuating one or more of actuation members 772, 774, a user may cause one or more electrodes 144, 146, 148, 150 to move radially outwardly from a central longitudinal axis of end effector 140, expanding end effector 140 and bringing one or more electrodes 144, 146, 148, 150 into engagement with an area of tissue. One or more pressure sensors (not shown) may be placed on electrodes 144, 146, 148, 150 to provide the user with feedback on the amount of pressure exerted by electrodes 144, 146, 148, 150 on the area of tissue. Locking mechanism 776 may hold end effector 140 in its expanded configuration even after the user releases handle actuation members 772, 774. When locking mechanism 776 is released, end effector 140 may contract from its expanded configuration. Locking mechanism 776 may include, for example, a ratchet mechanism that may releasably hold actuation members 772, 774 in one or more positions.

An electrosurgical unit (not shown) may supply electrical energy to a connector (not shown) in handle 136. The electrosurgical unit may include, for example, a radiofrequency unit. The electrical energy may be conducted from the connector to shaft 138, and then from shaft 138 to one or more electrodes 144, 146, 148, 150. One or more electrodes 144, 146, 148, 150 may perform therapy on an area of tissue by delivering the energy to the area of tissue.

One or more actuator assemblies 120, 122, 124, 126 may engage therapy device 106. For example, one or more actuator assemblies 120, 122, 124, 126 may engage sheath 134 at a proximal portion of sheath 134 outside of endoscope 104. One or more actuator assemblies 120, 122, 124, 126, or any combination of actuator assemblies 120, 122, 124, 126, may produce translational movement of therapy device 106 relative to body lumen 102. This may include movement of therapy device 106 towards and away from the tissue surface (e.g., heaving and/or swaying), and/or movement of therapy device 106 along the tissue surface (e.g., surging). Additionally or alternatively, one or more actuator assemblies 120, 122, 124, 126, or any combination of actuator assemblies 120, 122, 124, 126, may produce rotational movement of therapy device 106 relative to the tissue surface. This may include tilting of therapy device 106 relative to the tissue surface (e.g., pitching and/or yawing), and/or rotating of therapy device 106 about its longitudinal axis (e.g., rolling). One or more actuator assemblies 120, 122, 124, 126 may include driven rollers configured to produce one or more of the translational and/or rotational movements. One or more actuator assemblies 120, 122, 124, 126 may include one or more motors (not shown) to drive the rollers.

One or more sensor assemblies 108, 110, 112, 114, 116 may be coupled to therapy device 106. For example, sensor assembly 108 may be coupled to end cap 152. Sensor assembly 110 may be coupled to electrode 144. Sensor assembly 112 may be coupled to electrode 150. Sensor assembly 114 may be coupled to elongate member 134 at or near the distal end of elongate member 134. Sensor assembly 116 may be coupled to elongate member 134 further from the distal end of elongate member 134. It is contemplated that only one of sensor assemblies 108, 110, 112, 114, 116 may be present on therapy device 106. Alternatively, any combination of sensor assemblies 108, 110, 112, 114, 116 may be present on the same therapy device 106.

As shown in FIG. 1C, sensor assembly 108 may include a source 154, a detector 156, and a processor 158. Source 154 may be configured to emit energy, an electric field, and/or a magnetic field. For example, source 154 may include a light source, such as a light-emitting diode, laser diode, or any other suitable emitter of energy. Source 154 may emit light onto a tissue surface, such as a surface of body lumen 102. Source 154 may emit patterned light. For example, the light may be emitted in fringes. Additionally or alternatively, source 154 may produce coherent waves, such as sound waves and/or ultrasound waves, in place of or in addition to the light waves. It is also contemplated that source 154 may include a plurality of emitters to produce interference fringes on the tissue surface.

Detector 156 may be configured to detect emitted energy, an electric field, and/or a magnetic field from source 154. Detector 156 may generate a detector output signal indicative of one or more characteristics of the detected energy and/or field. For example, detector 156 may include a light detector, such as an array of photodiodes, one or more image sensors, or any other suitable receiver. The one or more image sensors may include, for example, an optoelectronic sensor and/or a video camera. Detector 156 may take images of a tissue surface (e.g., a surface of body lumen 102), off of which light from source 154 has been reflected. Detector 156 may image naturally occurring textural features of the surface. The textural features, when lit by source 154, may create regions of light and dark on the surface. Detector 156 may capture images of the surface in succession. Detector 156 may detect a Doppler frequency change and/or beats from a Doppler shift.

The detector output signal may be received by processor 158. Processor 158 may determine, based on the detector output signal, whether therapy device 106 has moved relative to the tissue surface. For example, processor 158 may receive the successive images captured by detector 156, and compare them with each other. The comparison may include comparing the regions of light and dark. Additionally or alternatively, the comparison may include cross-correlation, digital image correlation, differential digital image tracking, and/or any other suitable processing technique that can be used to determine a movement vector of therapy device 106 relative to the tissue surface. Through this comparison, processor 158 may determine whether source 154 and detector 156, and therefore therapy device 106 (e.g., the portions of therapy device 106 on which source 154 and/or detector 156 may be mounted), have moved relative to the surface. Processor 158 may also determine the magnitude and/or direction of the movement.

Movements that may be detected may include translational movement of therapy device 106 relative to the surface. This may include movement of therapy device 106 towards and away from the surface (e.g., heaving and/or swaying), and/or movement of therapy device 106 along the surface (e.g., surging). Additionally or alternatively, the movement that may be detected may include rotational movement of therapy device 106 relative to the surface. This may include tilting of therapy device 106 relative to the surface (e.g., pitching and/or yawing), and/or rotating of therapy device 106 about its longitudinal axis (e.g., rolling).

Alternatively, source 154 and detector 156 may be configured to function as a laser Doppler anemometer, a laser Doppler vibrometer, an ultrasound emitter and detector, Hall effect sensor and magnet, inductive coil and magnet, and/or any other suitable mechanism configured to detect relative motion between objects. Such mechanisms may be advantageous when used in areas of the body in which opaque fluids may present a barrier to light.

Source 154, detector 156, and processor 158 may be coupled to one another, and to one or more power sources, by any suitable wired or wireless electrical connections. For example, wiring for source 154 and detector 156 may run proximally along or within components of therapy device 106 to processor 158, which may be at a proximal end of sheath 134 or separated from therapy device 106.

An identification element 160 may be provided on the tissue surface. Additionally or alternatively, identification element 160 may be provided within the tissue. Identification element 160 may include one or more labels, markers, tags, fluorescent dye dots, and/or magnets that can be provided on or in the tissue. Additionally or alternatively, an identification element 162 may be provided on the tissue surface or in the tissue. Identification element 162 may include a plurality of identification elements 160. The plurality of identification elements 160 may be arranged to form a predetermined pattern or matrix. Identification elements 160, 162 may reflect energy emitted by source 154, and/or fields emitted by source 154. Additionally or alternatively, identification elements 160, 162 may emit energy and/or fields detectable by detector 156. It is contemplated that source 154 may work in conjunction with identification elements 160, 162, or alternatively, source 154 may be replaced by identification elements 160, 162. Identification element 160 and/or identification element 162 may be introduced on or in the tissue using a separate delivery catheter (not shown). Alternatively, the delivery catheter may be part of, or may be coupled to, therapy device 106.

Detector 156 may take images of one or more of identification elements 160, 162, from which light from source 154 has been reflected. Detector 156 may image one or more features of identification elements 160, 162. Detector 156 may capture images of identification elements 160, 162 in succession. Processor 158 may receive the successive images, and compare them with each other to determine how far source 154 and detector 156, and therefore therapy device 106, have moved relative to identification elements 160, 162, and therefore the tissue surface. In some instances, identification elements 160, 162 may be more easily and/or accurately detectable by detector 156 than tissue surface features.

After processor 158 determines how far therapy device 106 has moved relative to the tissue surface, processor 158 may generate one or more output signals to one or more actuator assemblies 120, 122, 124, 126. The one or more output signals from processor 158 may instruct one or more actuator assemblies 120, 122, 124, 126 to automatically move therapy device 106 in response to the determined movement of therapy device 106 relative to the tissue surface. For example, the one or more output signals may instruct one or more actuator assemblies 120, 122, 124, 126 to move therapy device 106 to counteract the determined movement of therapy device 106 relative to the tissue surface. Counteracting the determined movement may include cancelling out the determined movement by moving therapy device 106 back to its position on the tissue surface prior to occurrence of the determined movement. Alternatively, counteracting the determined movement may include mitigating the determined movement by moving therapy device 106 within a predetermined range of its position on the tissue surface prior to occurrence of the determined movement. It is contemplated that the one or more output signals may be generated such that one or more actuator assemblies 120, 122, 124, 126 may begin counteracting the determined movement as soon as the determined movement is detected, to limit the magnitude of the determined movement. One result may be that relative motion between end effector 140 may be minimized or eliminated, with end effector 140 being moved by one or more actuator assemblies 120, 122, 124, 126 in synchronization with the tissue surface.

Body motions caused by heartbeats and/or breathing may be periodic in nature, and may be repetitive. Processor 158 may include a control algorithm configured to learn the periodic repeated motions, and predict the motions that may compensate for future periodic repeated motions. The predicted compensating motions may be implemented by processor 158 and actuator assemblies 120, 122, 124, 126 in real time, e.g., simultaneously with the occurrence of the periodic repeated motions. It is also contemplated that finer motion compensation may be provided by feedback supplied from source 154, detector 156, and processor 158, to compensate for variability in the periodic repeated motions.

Additionally or alternatively, the one or more output signals may instruct one or more actuator assemblies 120, 122, 124, 126 to move end effector 140 predetermined distances, and/or to predetermined locations, along the tissue surface. For example, the one or more output signals may instruct one or more actuator assemblies 120, 122, 124, 126 to move end effector 140 to a position on the tissue surface so that therapy device 106 may deliver therapy at that position. The one or more output signals may also instruct one or more actuator assemblies 120, 122, 124, 126 to move therapy device 106 to a plurality of positions on the tissue surface. Therapy may be delivered at each of those predetermined positions.

It is contemplated that one or more actuator assemblies 120, 122, 124, 126 may move end effector 140 in one or more modes. For example, one or more actuator assemblies 120, 122, 124, 126 may move end effector 140 in a first mode in which one or more rough movements are performed. Rough movements may include movements of large magnitude that may be performed at a rapid pace. One or more actuator assemblies 120, 122, 124, 126 may move end effector 140 in a second mode in which one or more fine movements are performed. Fine movements may include movements of smaller magnitude that may be performed at a slower pace. The first mode may be used to bring end effector 140 close to a position of body lumen 102. The second mode may be used to bring end effector 140 to the position, so that electrodes 144, 146, 148, 150 may be positioned with greater accuracy than the accuracy associated with the first mode and/or manual positioning by a user. Increasing the accuracy of positioning of electrodes 144, 146, 148, 150 may increase the effectiveness of the therapy.

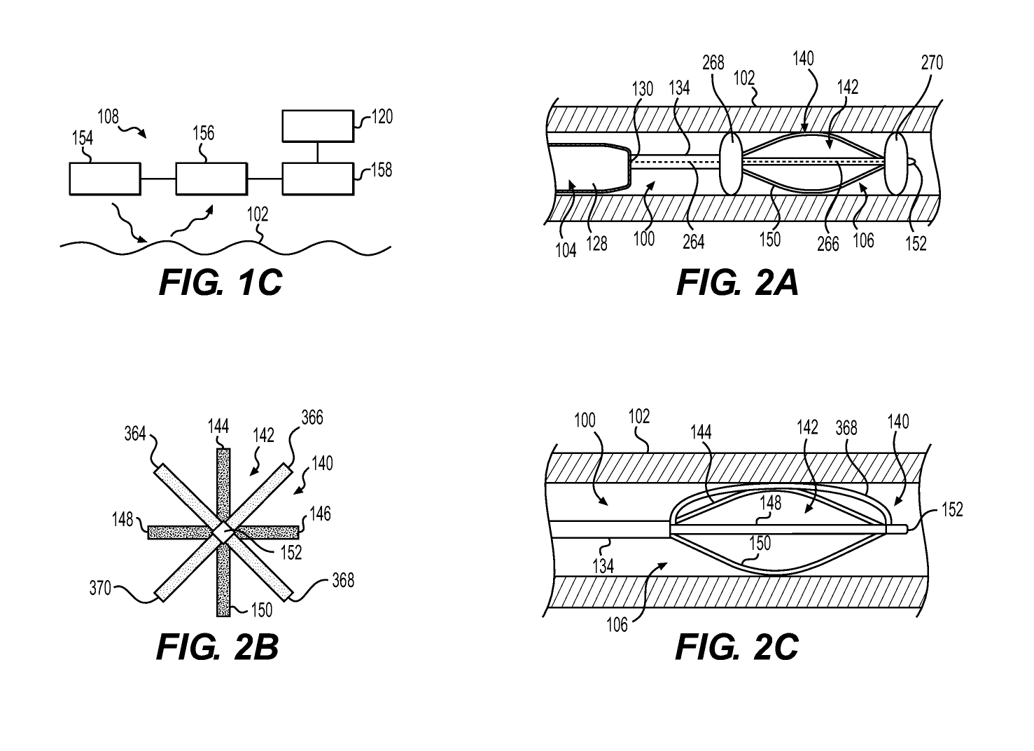

As illustrated in FIGS. 2A-2F, apparatus 100 may include one or more features to help stabilize end effector 140 relative to an area of tissue. In FIG. 2A, end effector 140 may include one or more expandable members 268, 270. Expandable members 268, 270 may include inflatable balloons. Expandable members 268, 270 may be inflated by using inflation lumens 264, 266 to direct an inflation fluid, such as one or more gases (e.g., air), from a fluid source (not shown) into expandable members 268, 270, causing them to expand radially outwardly. Alternatively, expandable members 268, 270 may be self-expanding such that expansion may be achieved without requiring the use of an inflation fluid. For example, expandable members 268, 270 may be spring-biased to expand radially outwardly. Expandable members 268, 270 may contact areas of tissue. That contact may help stabilize electrode array 142 of end effector 140 relative to an area of tissue.

In FIGS. 2B and 2C, one or more non-conducting legs 364, 366, 368, 370 may be provided on end effector 140. Non-conducting legs 364, 366, 368, 370 may differ from electrodes 344, 346, 348, 350 in shape, size, or any other characteristic. Through their contact with an area of tissue, non-conducting legs 364, 366, 368, 370 may increase frictional engagement between the area of tissue and end effector 140, thereby helping to stabilize end effector 140 relative to the area of tissue. Any suitable number of non-conducting legs may be provided. The number of non-conducting legs need not equal the number of electrodes.

In FIG. 2D, one or more electrodes 144, 146, 148, 150 may include one or more roughened portions and/or protrusions 468. One or more roughened portions and/or protrusions 468 may increase frictional contact between electrodes 144, 146, 148, 150 with an area of tissue. The increased frictional contact may help stabilize electrodes 144, 146, 148, 150 relative to the area of tissue.

In FIG. 2E, a strain-relief member 568 may be at and/or coupled to a distal end of elongate member 134. End effector 140 may be at and/or coupled to a distal end of strain-relief member 568. Strain-relief member 568 may allow end effector 140 to move relative to elongate member 134. If end effector 140 is in contact with an area of tissue, and the area of tissue moves, strain-relief member 568 may extend distally, retract proximally, bend laterally, and/or twist, to help maintain frictional contact between electrodes 144, 146, 148, 150 with the area of tissue.

It is contemplated that strain-relief member 568 may be constrained to have a substantially straight configuration by surfaces of working channel 130 when strain-relief member 568 is within working channel 130. Strain-relief member 568 may move into a bent or coiled configuration upon exiting working channel 130. Strain-relief member 568 may be less flexible when it is in its straight configuration than when it is in its bent configuration. Thus, when it is in its straight configuration, strain-relief member 568 may transmit translational and/or rotational forces, exerted by a user on the proximal end of elongate member 134, to end effector 140. When it is in its bent configuration, strain relief member 568 may take up (not transmit) translational and/or rotational forces between end effector 140 and elongate member 134. An optional fiber 570 may be provided to allow a user to pull end effector 140 proximally after strain-relief member 568 and end effector 140 have exited from working channel 130, in case such movement of end effector 140 is desired. Fiber 570, which may be a wire, cable, or other elongate member, may extend to the proximal end of elongate member 134 where it may be gripped by the user.

In FIG. 2F, end effector 140 may include an expandable member 668. Expandable member 668 may include an inflatable balloon. Expandable member 668 may be inflated by fluid, such as one or more gases (e.g., air), supplied from a fluid source (not shown) via an inflation lumen 670. Additionally or alternatively, expandable member 668 may be self-expandable. For example, expandable member 668 may be spring-biased to move radially outwardly in the absence of a constraining force. Expandable member 668 may exert a radially outward force on one or more electrodes 144, 146, 148, 150 to increase frictional contact of electrodes 144, 146, 148, 150 with an area of tissue, thereby helping to stabilize them relative to the area of tissue. Additionally or alternatively, expandable member 668 may prevent one or more electrodes 144, 146, 148, 150 from inverting from a convex curved configuration to a concave curved configuration, due to radially-inward forces exerted on one or more electrodes 144, 146, 148, 150 by the tissue.

As shown in FIG. 3, locking device 776 may maintain end effector 140 in its expanded configuration even after a user releases actuation members 772, 774. This may allow end effector 140 to move with the area of tissue more freely than if the user was holding handle 136, thereby helping to stabilize end effector 140 relative to the area of tissue. It is also contemplated that elongate member 134 may be provided with a smooth outer surface and/or coating with a lubricious material to allow elongate member 134 to slide against tissue, and thereby help maintain frictional engagement between end effector 140 and the body lumen.

It is contemplated that the stabilization features shown in FIGS. 2A-2F and 3 may be used individually or in any suitable combination, to help stabilize end effector 140. It is also contemplated that one or more of electrodes 144, 146, 148, 150, one or more of non-conducting legs 364, 366, 368, 370, and/or strain-relief member 568, may be made of a shape-memory material such as Nitinol. Such components may be held in a compressed configuration during delivery through working channel 130, and may move to an expanded configuration upon after exiting from the working channel.

While a basket-type end effector is illustrated in FIGS. 1A and 2A-2F, it should be understood that end effector 140 may include another type of device. The device may include, for example, a biopsy instrument (not shown). The biopsy instrument may be a forceps, snare, core needle, or any other suitable instrument for removing tissue for examination. The biopsy instrument may be at and/or coupled to the distal end of elongate member 134. The biopsy instrument may be coupled to the distal end of shaft 138, and may be extended distally out of and/or retracted proximally into elongate member 134. Actuation members 772, 774 may be actuated to cause biopsy instrument to open and/or close to remove tissue.

Alternatively, the device may include a valve delivery device, such as a device for delivering a transcatheter aortic or other heart valve (not shown). The valve may be at and/or coupled to the distal end of elongate member 134. The valve may be coupled to the distal end of shaft 138. The valve may be removably coupled to elongate member 134 or shaft 138, allowing the valve to be guided to a target area using elongate member 134 or shaft 138, and then deployed at the target area and left there as elongate member 134 or shaft 138 is removed. Actuation member 772, 774 may be actuated to deploy the valve. It is also contemplated that a stent (not shown) may be delivered in a manner similar to the valve.

One or more of sensor assemblies 108, 110, 112, 114, 116 (FIG. 1A) may be mounted on the biopsy instrument, valve, stent, and/or device. The biopsy instrument or valve may be positioned relative to an area of tissue, and stabilized in that position, in the same manner as that which has been described above with respect to end effector 140. This may help ensure that a biopsy is performed on a particular area of tissue, or that the transcatheter aortic valve or stent is properly positioned at a particular area of tissue, in spite of the area of tissue moving due to a patient's breathing and/or heartbeat.

Methods of performing therapy on areas of tissue will now be described. Therapy device 106 may be inserted through working channel 130 of endoscope 104 by sliding therapy device 106 distally relative to endoscope 104. During insertion, the user may not actuate actuation members 772, 774 of handle 136. End effector 140 may be in a contracted, unactuated, or undeployed configuration.

Therapy device 106 may be advanced distally through working channel 130 until end effector 140, a distal portion of elongate member 134, and/or a distal portion of shaft 138 may extend distally out of working channel 130 at the distal end of endoscope 104. End effector 140, a distal portion of elongate member 134, and/or a distal portion of shaft 138 may be positioned in view of imaging device 132 of endoscope 104.

End effector 140 may be advanced to a target area of tissue. End effector 140 may be advanced manually, at least part of the way, under endoscopic vision provided by imaging device 132. Additionally or alternatively, visual features of the area of tissue and/or visual features of one or more identification elements 160, 162 may be read by one or more sensor assemblies 108, 110, 112, 114, 116. Based on the readings, one or more actuator assemblies 120, 122, 124, 126 may move therapy device end effector 140 in the first mode in which one or more rough movements are performed. Additionally or alternatively, one or more actuator assemblies 120, 122, 124, 126 may move end effector 140 in the second mode in which one or more fine movements are performed. The rough movements and/or the fine movements may be performed to position end effector 140 at the area of tissue, and/or to reposition end effector 140 at the area of tissue if the area of tissue moves relative to end effector 140 due, for example, to a patient's breathing, heartbeat, or any other such movement. When actuator assemblies 120, 122, 124, 126 move end effector 140, handle 136 may be released by the user to prevent the user from impeding proper positioning of therapy device 106.

Once end effector 140 has been positioned at the target area, end effector 140 may be expanded, actuated, or deployed. This may be achieved when the user actuates actuation members 772, 774. Alternatively, end effector 140 may be partially expanded, actuated, or deployed during positioning, and then fully expanded, actuated, or deployed once the target area has been reached.

When electrode array 142 has been expanded, electrodes 144, 146, 148, 150 may engage the target area of tissue. For example, electrodes 144, 146, 148, 150 may contact walls of a body lumen 102, such as a lung airway. The user may actuate a switch (not shown) to deliver electrical energy to the area of tissue. The controller may be programmed to deliver the energy automatically according to preset parameters for time, energy, power, and temperature. Alternatively, when the biopsy instrument has been actuated, it may remove tissue from the target area. Alternatively, when the valve has been deployed, it may engage tissue at the target area.

If the area of tissue moves relative to electrode array 142 during delivery of the energy, the movement may be sensed by one or more sensor assemblies 108, 110, 112, 114, 116, resulting in one or more actuator assemblies 120, 122, 124, 126 moving electrode array 142 to compensate for, or cancel out, the movement.

Additionally or alternatively, based on readings from one or more sensor assemblies 108, 110, 112, 114, 116, one or more actuator assemblies 120, 122, 124, 126 may move electrode array 142 to another area of tissue. The movements may be rough and/or fine. Electrode array 142 may delivery energy to the other area of tissue. This may be repeated to delivery energy to the tissue at predetermined areas to create a predetermined pattern of areas treated by the energy. Alternatively, similar processes may be utilized with the biopsy device to keep the biopsy device at or near the area of tissue to be removed, and/or to reposition the biopsy device to take one or more tissue biopsies at predetermined areas. Alternatively, similar processes may be utilized with the transcatheter aortic valve to keep the valve at or near the valve site during deployment, so the valve can push native valve leaflets out of the way and take over the job of regulating blood flow.

The disclosed apparatuses and methods may be utilized in any suitable application involving the positioning of devices relative to areas of tissue in the body. Any aspect set forth herein may be used with any other aspect set forth herein. The apparatuses may be used in any suitable medical procedure, and may be advanced through any suitable body lumen and body cavity.

It will be apparent to those skilled in the art that various modifications and variations can be made in the disclosed apparatuses and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. An apparatus for positioning a device relative to tissue, comprising:
an emitter coupled to the device, the emitter being configured to emit energy onto the tissue,
a detector coupled to the device, the detector being configured to detect energy reflected from the tissue and generate a detector output signal indicative of a characteristic of the reflected energy, and
a processor configured to receive the detector output signal from the detector, determine whether there has been relative movement between the device and the tissue based on the detector output signal, and generate a processor output signal based on the relative movement; and
an actuator assembly engaging the device, the actuator assembly being configured to receive the processor output signal from the processor, and move the device based on the processor output signal.

2. The apparatus of claim 1, wherein the emitter comprises a source of light configured to project light onto the tissue.

3. The apparatus of claim 1, wherein the detector comprises an imaging sensor configured to detect the energy reflected off of the tissue, and the detector output signal is indicative of a characteristic of the energy reflected off of the tissue.

4. The apparatus of claim 1, wherein the detector output signal is one of a plurality of detector output signals, the detector output signals comprising:
a first detector output signal indicative of a characteristic of the energy reflected off of the tissue at a first point in time, and
a second detector output signal indicative of a characteristic of the energy reflected off of the tissue at a second point in time.

5. The apparatus of claim 4, wherein the processor is configured to receive the first and second detector output signals, and determine whether there has been relative movement between the device and the tissue by comparing the first and second detector output signals.

6. The apparatus of claim 1, wherein the actuator assembly is configured to move the device to compensate for the relative movement between the device and the tissue.

7. The apparatus of claim 1, wherein the actuator assembly is configured to move the device in synchronization with movement of the tissue.

8. An apparatus, comprising:
a sheath having a lumen;
a device comprising:
a member having a proximal portion and a distal portion, the member being configured for insertion into the lumen of the sheath,
an end effector at the distal portion of the member,
an emitter configured to emit energy, and
a detector configured to detect reflected energy and generate a detector output signal indicative of a characteristic of the reflected energy;
a processor configured to receive the detector output signal from the detector, determine whether the end effector has moved relative to adjacent tissue based on the detector output signal, and generate a processor output signal based on the movement; and
an actuator assembly engaging the member, the actuator assembly being configured to receive the processor output signal from the processor, and move the device based on the processor output signal.

9. The apparatus of claim 8, wherein the end effector comprises an array of electrically-conductive legs configured to delivery electrical energy to tissue.

10. The apparatus of claim 9, wherein the end effector comprises at least one of an inflatable balloon, a plurality of protrusions on at least one of the electrically-conductive legs, and a non-conductive leg.

11. The apparatus of claim 8, wherein the end effector comprises one of a biopsy instrument, a valve, and a stent.

12. The apparatus of claim 8, wherein the actuator assembly is configured to compensate for movement of the device.

13. The apparatus of claim 8, wherein at least one of the emitter and the detector is mounted on an external surface of the device.

14. The apparatus of claim 8, wherein the emitter is configured to emit energy onto a surface, the detector is configured to detect the emitted energy after reflection off of the surface, and the actuator assembly is configured to synchronize movement of the device with movement of the surface.

15. A method for positioning a device relative to tissue, comprising:
emitting energy toward the tissue with an emitter;
detecting reflected energy from the tissue with a detector, and generating a detector output signal indicative of a characteristic of the reflected energy;
receiving the detector output signal at a processor;
determining a position of the device relative to the tissue based on the detector output signal using the processor;
generating a processor output signal with the processor based on the position;
receiving the processor output signal at an actuator assembly; and
moving the device relative to the tissue with the actuator assembly based on the processor output signal.

16. The method of claim 15, wherein determining the position of the device relative to the tissue comprises identifying relative movement between the device and the tissue.

17. The method of claim 15, wherein moving the device relative to the tissue with the actuator assembly comprises moving the device in synchronization with the tissue.

18. The method of claim 15, wherein moving the device relative to the tissue comprises moving the device with the actuator assembly in first and second modes, the first mode including a first movement performed at a greater speed than a second movement performed in the second mode.

19. The method of claim 15, wherein emitting energy toward the tissue comprises emitting energy toward an identification element on a surface of the tissue, and detecting the reflected energy comprises receiving the reflected energy after reflection off of the identification element.

20. The method of claim 15, wherein moving the device relative to the tissue comprises automatically moving the device relative to the tissue in a predetermined pattern using the actuator assembly.
